(19) United States
(12) Patent Application Publication
(10) Pub. No.: US 2026/0033735 A1
(43) Pub. Date: Feb. 5, 2026

(54) BIOIMPEDANCE MEASUREMENT CIRCUIT

(71) Applicant: ams Sensors Germany GmbH, Jena (DE)

(72) Inventors: Dalibor STOJKOVIC, Erfurt (DE); Julio SALDAN, Jena (DE); Parvathy SASIKALA JAYACHANDRAN PILLAI, Erfurt (DE)

(21) Appl. No.: 19/100,097

(22) PCT Filed: Jul. 24, 2023
(86) PCT No.: PCT/EP2023/070415

§ 371 Date: Jan. 30, 2025

(30) Foreign Application Priority Data
Aug. 1, 2022 (DE) 10 2022 119 225.3
Sep. 9, 2022 (DE) 10 2022 122 957.2

(51) Int. Cl. A61B 5/053; A61B 5/00

(52) U.S. Cl. CPC A61B 5/053; A61B 5/7271

(57) ABSTRACT

A bioimpedance measurement circuit comprises an electrode arrangement for attaching to a body, an evaluation circuit for determining a real part and an imaginary part of a bioimpedance of the body in response to a stimulus current applied to the body and an input voltage of the evaluation circuit. A signal processing circuit is configured to determine a corrected value of the bioimpedance by calculating a corrected real part of the bioimpedance in dependence on a derivative of the real part of the bioimpedance, and a corrected imaginary part of the bioimpedance in dependence on the imaginary part and a second imaginary part of the bioimpedance determined by the evaluation circuit at different measurement frequencies.

### TECHNICAL FIELD

The disclosure relates to a bioimpedance measurement circuit for measuring a bioimpedance across measurement electrodes.

### BACKGROUND

Bioimpedance measurement is a biomedical technique to determine the electrical behavior of living tissue by using an electrode arrangement for applying a stimulus current to a sample, for example a part of a human body, and measure the resulting voltage through a four point measurement method. The amplitude and phase of the resulting voltage signal will depend on the BIOZ impedance. The bioimpedance of interest may be determined by the measured voltage divided by the stimulus current injected into the tissue.

Due to asymmetries in a current path of the arrangement of the measuring electrodes to perform the four point measurement method, a common mode signal is created across the body impedance. Those asymmetries arise because of a mismatch between the skin/electrode contact impedances. Moreover, due to asymmetries in the voltage path between the measuring electrodes and a differential amplifier to evaluate the voltage across the measuring electrodes, the common mode signal is translated to an extra differential signal that is added to the desired differential signal. As a result, the measured magnitude of the total input differential signal which is applied to the differential amplifier is higher than the expected one and the phase is also affected.

There is a desire to provide a bioimpedance measurement circuit which provides the bioimpedance of interest as accurately as possible, and in particular attenuates the unwanted extra differential signal caused by the common mode signal across the bioimpedance.

### SUMMARY

A bioimpedance measurement circuit that attenuates the added unwanted differential signal and thus provides the bioimpedance of interest with high accuracy is specified in claim 1.

The bioimpedance measurement circuit comprises an electrode arrangement for attaching to a body, an evaluation circuit for determining a real part and an imaginary part of a bioimpedance of the body in response to a stimulus current applied to the body and an input voltage of the evaluation circuit. The measurement circuit further comprises a signal processing circuit for determining a corrected value of the bioimpedance. The electrode arrangement includes a first pair of electrodes to apply the stimulus current to the body, and a second pair of electrodes to measure the input voltage in dependence on a voltage drop across the bioimpedance between the electrodes the second pair of the electrodes.

The signal processing circuit is configured to determine the corrected value of the bioimpedance by calculating a corrected real part of the bioimpedance in dependence on a derivative of the real part of the bioimpedance, and a corrected imaginary part of the bioimpedance in dependence on the imaginary part and a second imaginary part of the bioimpedance determined by the evaluation circuit at different measurement frequencies.

In conclusion, the proposed solution is to attenuate the added unwanted differential signal by taking into account its frequency dependence and for cases when the measured impedance has dominant real part with weak frequency dependence. The desired differential signal for the bioimpedance to be measured has real part, imaginary part and magnitude that in general decrease with frequency. The undesired differential signal has real part, imaginary part and magnitude that increase with the frequency around the typical excitation frequency, for example 50 KHz. The evaluation circuit may be configured to determine the real part and the imaginary part of the bioimpedance by quadrature demodulation.

The proposed bioimpedance measurement circuit allows to correct the measured real and imaginary part of the bioimpedance without the need of any extra hardware components, especially on the device level, i.e. no additional circuits or calibration pins are necessary. Moreover, the circuit designs can be simplified so that, as a result, the product costs are not increased.

According to an embodiment of the bioimpedance measurement circuit, the signal processing circuit is configured to determine the corrected real part of the bioimpedance by subtracting a first correction term from the real part of the bioimpedance. According to a possible embodiment of the bioimpedance measurement circuit, the signal processing circuit is configured to determine the first correction term in dependence on the derivative of the real part of the bioimpedance.

That means, for the real part of the bioimpedance to be corrected, based on the assumption of weekly frequency dependence real part, it is proposed to estimate the derivative of the real part and from that reconstruct the undesired real part contribution.

According to a further embodiment of the bioimpedance measurement circuit, the signal processing circuit is configured to determine the corrected imaginary part of the bioimpedance by subtracting a second correction term from the imaginary part of the bioimpedance. According to a possible embodiment of the measurement circuit, the signal processing circuit is configured to determine the second correction term in dependence on the second imaginary part of the bioimpedance.

For the imaginary part of the bioimpedance to be corrected, based on the assumption that the impedance is mostly real and the small imaginary part decreases with frequency, it is thus proposed to measure the second imaginary part at another frequency than the frequency of interest, for example at some high frequency, i.e. higher than the frequency of interest, and then this measured second imaginary part will be a good approximation of the undesired imaginary part.

According to an embodiment, the bioimpedance measurement circuit comprises a control circuit to control a measurement of the bioimpedance. The control circuit is configured to control the determination of the corrected value of the bioimpedance by carrying out at least four measurements of the bioimpedance at four different measurement frequencies.

According to a possible embodiment of the bioimpedance measurement circuit, the circuit comprises a controllable stimulus current generator to apply the stimulus current to the electrode arrangement with the at least four different measurement frequencies.

According to an embodiment of the bioimpedance measurement circuit, the control circuit is configured to control the controllable stimulus current generator so that the stimulus current is applied to the electrode arrangement with a first one of the measurement frequencies during a first one of the at least four measurements of the bioimpedance. The evaluation circuit is configured to determine a second real part of the bioimpedance during the first measurement of the bioimpedance.

According to a possible embodiment of the bioimpedance measurement circuit, the control circuit is configured to control the controllable stimulus current generator so that the stimulus current is applied to the electrode arrangement with a second one of the measurement frequencies during a second one of the at least four measurements of the bioimpedance. The evaluation circuit is configured to determine a third real part of the bioimpedance during the second measurement of the bioimpedance.

The first and second measurement is performed to determine the derivative of the real part of the bioimpedance in dependence on the second real part of the bioimpedance and the third real part of the bioimpedance, and thus to determine the first correction term which is is used to calculate the corrected real part of the bioimpedance.

According to a possible embodiment of the bioimpedance measurement circuit, the signal processing circuit is configured to determine the derivative of the real part of the bioimpedance by determining a quotient of the difference of the second and third real part of the bioimpedance, and the difference of the first and second measurement frequencies. The derivative can thus be estimated by subtracting two measurements at two consecutive frequencies among the available frequencies.

According to a possible embodiment of the bioimpedance meausurement circuit, the control circuit is configured to control the controllable stimulus current generator so that the stimulus current is applied to the electrode arrangement with a third one of the measurement frequencies during a third one of the at least four measurements of the bioimpedance. The evaluation circuit is configured to determine the second imaginary part of the bioimpedance during the third measurement of the bioimpedance.

The third measurement is performed to determine the second imaginary part, and thus to determine the second correction term which is is used to calculate the corrected imaginary part of the bioimpedance.

According to an embodiment of the bioimpedance measurement circuit, the control circuit is configured to control the controllable stimulus current generator so that the stimulus current is applied to the electrode arrangement with a fourth one of the measurement frequencies during a fourth one of the at least four measurements of the bioimpedance. The evaluation circuit is configured to determine the real part and the imaginary part of the bioimpedance during the fourth measurement of the bioimpedance.

According to an embodiment of the bioimpedance measurement circuit, the control circuit is configured to control the controllable stimulus current generator so that the first and second frequencies are closer to the fourth frequency, i.e. the frequency of interest, than the third frequency. The third frequency is higher than the fourth frequency.

According to a possible embodiment of the bioimpedance measurement circuit, the signal processing circuit is configured to determine the corrected real part of the bioimpedance by subtracting the first correction term from the real part of the bioimpedance during the fourth measurement. The signal processing circuit is configured to determine the corrected imaginary part of the bioimpedance by subtracting the second correction term from the imaginary part of the bioimpedance during the fourth measurement.

Additional features and advantages of the bioimpedance measurement circuit are set forth in the detailed description that follows. It is to be understood that both the foregoing general description and the following detailed description are merely exemplary, and are intended to provide an overview or framework for understanding the nature and character of the claims.

### BRIEF DESCRIPTION OF THE DRAWINGS

The accompanying drawings are included to provide further understanding, and are incorporated in, and constitute a part of, the specification. As such, the disclosure will be more fully understood from the following detailed description, taken in conjunction with the accompanying figures in which:

FIG. 1 shows a bioimpedance measurement circuit with asymmetries in the current/voltage paths because of mismatch between the skin/electrode contact impedances;

FIG. 2 illustrates a simplified model to explain the common mode to differential mode translation; and

FIG. 3 shows an embodiment of a bioimpedance measurement circuit with attenuation of an unwanted differential signal caused by a common mode signal.

### DETAILED DESCRIPTION OF THE EMBODIMENTS

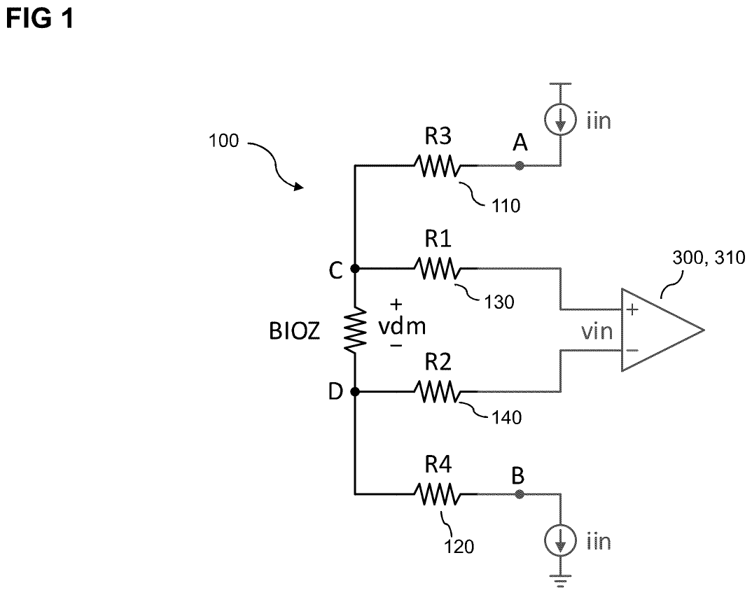

FIG. 1 illustrates a bioimpedance measurement circuit that is based on a four point measurement method. The bioimpedance measurement circuit comprises an an electrode arrangement 100 for attaching to a human body. The electrode arrangement 100 includes a first pair of electrodes 110, 120 to apply a stimulus current iin to the body. The electrodes 110, 120 are attached to nodes A and B of a human body. It is assumed that electrode 110 has an electrode contact impedance R3, and electrode 120 has an electrode contact impedance R4. The electrode arrangement further comprises a second pair of electrodes 130, 140 to measure a voltage vin in dependence, inter alia, on a voltage drop vdm across a bioimpedance BIOZ of the body between the electrodes 130, 140 of the second pair. The electrodes 130, 140 are attached to nodes C and D of a human body. It is assumed that electrode 130 has an electrode contact impedance R1, and electrode 140 has an electrode contact impedance R2.

The electrodes 110, 120, 130 and 140 are placed to measure the bioimpedance BIOZ of interest between nodes C and D of the human body or between electrodes 130 and 140. The generated voltage vin is applied to and evaluated by an evaluation circuit 300 which is illustrated in simplified form as a differential amplifier 310 in FIG. 1. The amplitude and phase of the resulting voltage signal vin depends on the bioimpedance BIOZ of interest, and the bioimpedance BIOZ is the quotient vin/iin.

Ideally, the voltage vin at the input of the evaluation circuit 300 is equal to the desired differential signal vdm which represents the voltage drop across the bioimpedance BIOZ. In reality, however, due to asymmetries in the current path a common mode signal is created across the body impedance BIOZ. Assuming that in some implementations, the common mode at nodes A and B is regulated to be some internal defined reference voltage vref, the common mode signal across nodes C and D is:

$$\frac{VC + VD}{2} = \frac{VA + VB}{2} + \frac{R 4 - R 3}{2} \cdot iin = vref + k \cdot iin$$

where VA is the voltage potential at node A, VB is the voltage potential at node B, VC is the voltage potential at node C, and VD is the voltage potential at node D.

As a result, a common mode signal vcm is generated across the bioimpedance BIOZ between nodes C and D, wherein the common mode signal vcm is proportional to the stimulus current:

$$vcm = k \cdot iin$$

Due to asymmetries in the voltage path, the common mode signal vcm is translated to an unwanted extra differential signal vdmx. Those asymmetries arise because of mismatch between the skin/electrode contact impedances.

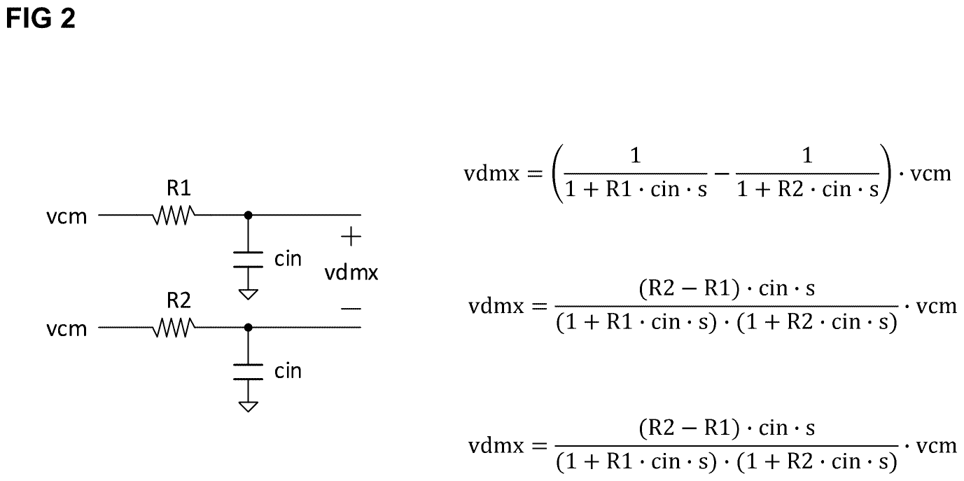

FIG. 2 shows a simplified model to explain the common mode to differential mode translation.

The differential signal vdmx which comes from the translation of the common mode signal vcm is added to the desired differential signal vdm. Then the total measured signal is:

$$vin = vdm + vdmx$$

where vin is the total input differential signal, vdm is the desired differential signal, related to the impedance to be measured, and vdmx is the undesired differential signal, coming from the translation from the common mode signal to differential mode signal.

As a result the measured magnitude is higher than the expected one and the phase is also affected.

Referring to FIG. 2, the undesired differential signal vdmx caused by the translation of the common mode signal is or

$$\begin{matrix}vdmx = \frac{( R 2 - R 1 ) \cdot cin \cdot j \cdot ω}{( 1 + R 1 \cdot cin \cdot j \cdot ω ) \cdot ( 1 + R 2 \cdot cin \cdot j \cdot ω )} \cdot vcm \\ vdmx = \frac{( R 2 - R 1 ) \cdot cin \cdot j \cdot ω}{( 1 + ( R 1 + R 2 ) \cdot cin \cdot j \cdot ω - R 1 \cdot R 2 \cdot {cin}^{2} \cdot {ω}^{2} )} \cdot vcm\end{matrix}$$

Considering typical values for the electrode contact impedances R1=5 kΩ, R2=15 kΩ, and cin=10 pF, and a frequency of interest of the stimulus current F=50 kHz, the second term of the real part of the dominator is

$$R 1 \cdot R 2 \cdot {cin}^{2} \cdot {ω}^{2} = 0.00074 ≪ 1$$

and the imaginary part of the dominator is

$$( R 1 + R 2 ) \cdot cin \cdot ω = 0.06283$$

Then, for values in the same order of magnitude the expression of the undesired differential signal vdmx can be simplified to:

$$\begin{matrix}vdmx = \frac{( R 2 - R 1 ) \cdot cin \cdot j \cdot ω}{( 1 + ( R 1 + R 2 ) \cdot cin \cdot j \cdot ω )} \cdot vcm \\ = \frac{( R 2 - R 1 ) \cdot cin \cdot j \cdot ω}{( 1 + ( r 1 + R 2 ) \cdot cin \cdot j \cdot ω )} \cdot \frac{( 1 - ( R 1 + R 2 ) \cdot cin \cdot j \cdot ω )}{( 1 - ( R 1 + R 2 ) \cdot cin \cdot j \cdot ω )} \cdot vcm \\ = \frac{( R {2}^{2} - R {1}^{2} ) \cdot {cin}^{2} \cdot {ω}^{2} + ( R 2 - R 1 ) \cdot cin \cdot j \cdot ω}{1 - {( R 1 + R 2 )}^{2} \cdot {cin}^{2} \cdot {ω}^{2}} \cdot vcm\end{matrix}$$

For the same set of values as in the previous example, the second term in the denominator is: 0.06283²=0.00395<<1 As a result, the term denoting the differential signal can be further simplified to:

$$vdmx = [ ( R {2}^{2} - R {1}^{2} ) \cdot {cin}^{2} \cdot {ω}^{2} + ( R 2 - R 1 ) \cdot cin \cdot j \cdot ω ] \cdot vcm$$

It is concluded that both the real part and imaginary part have the same sign, and their absolute values increase with frequency. The validity of that approximation is restricted to frequencies where the assumptions made above are valid. The proposed correction method is based on that approximation, however other corrections can be also proposed by including all the terms in the expression. For better approximation, valid in wider range of frequencies, the resulting correction method will be more complex.

The total measured voltage will be:

$$vin = vdm + vdmx$$

and thus

$$vin = iin \cdot [ a ( ω ) + j \cdot b ( ω ) ] + [ ( R {2}^{2} - R {1}^{2} ) \cdot {cin}^{2} \cdot {ω}^{2} + ( R 2 - R 1 ) \cdot cin \cdot j \cdot ω ] \cdot vcm$$

Considering the the common mode signal is proportional to the applied current: vcm=k·iin, the input voltage at the input side of the differential amplifier 310 is

$$vin = iin \cdot [ a ( ω ) + j \cdot b ( ω ) + k \cdot ( R {2}^{2} - R {1}^{2} ) \cdot {cin}^{2} \cdot {ω}^{2} + k \cdot ( R 2 - R 1 ) \cdot cin \cdot j \cdot ω ]$$

The real part I of the bioimpedance BIOZ is:

$$I = Re ( BIOZ ) = a ( ω ) + k \cdot ( R {2}^{2} - R {1}^{2} ) \cdot {cin}^{2} \cdot {ω}^{2}$$

and the imaginary part Q of the bioimpedance is:

$$Q = Im ( BIOZ ) = b ( ω ) + k \cdot ( R 2 - R 1 ) \cdot cin \cdot ω$$

The idea is to extract the respective portion of the real part I and the imaginary part Q generated by the unwanted common mode signal, which are written ‘bold’ in the formulas.

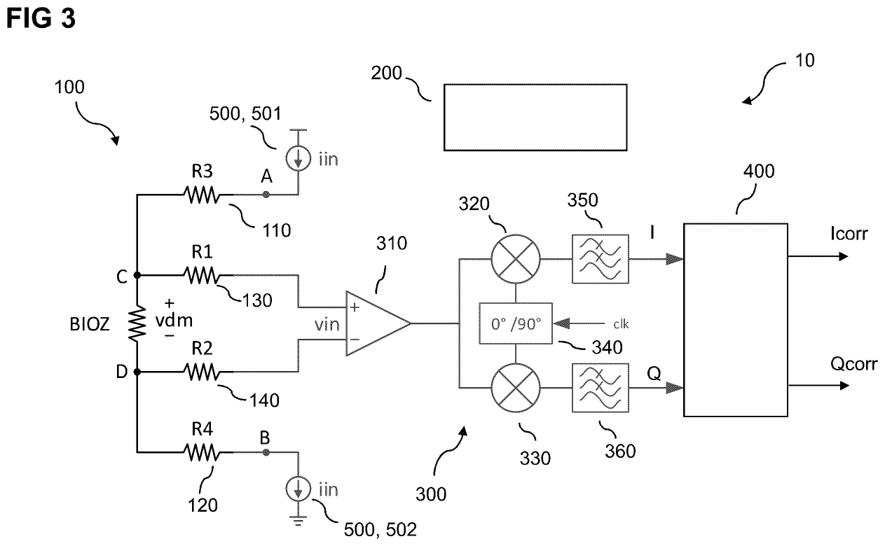

A bioimpedance measurement circuit 10 which allows to determine the real part I and the imaginary part Q of a measured bioimpedance is shown in FIG. 3. The measurement circuit 10 comprises an electrode arrangement 100 for attaching to a body. The electrode arrangement 100 includes a first pair of electrodes 110, 120 to apply a stimulus current iin to the body, and a second pair of electrodes 130, 140 to measure an input voltage vin in dependence on a voltage drop across a bioimpedance BIOZ between the electrodes 130, 140 of the second pair of the electrodes. The electrodes 110, 120 are attached to nodes A and B of a human body, wherein R3 denotes an electrode contact impedance between the skin of the body and the electrode 110, and R4 denotes an electrode contact impedance between the skin of the body and the electrode 120.

The bioimpedance measurement circuit 10 further comprises a control circuit 200 to control a measurement of the bioimpedance BIOZ, and an evaluation circuit 300. The evaluation circuit 300 is configured for determining a real part I and an imaginary part Q of the bioimpedance BIOZ in response to the stimulus current iin and the measured input voltage vin applied to the evaluation circuit 300.

The evaluation circuit 300 comprises a differential amplifier 310 to apply the measured input voltage vin. An output side of the differential amplifier 310 is coupled to a first path comprising a modulator 320 and a low pass filter 350, and a second path comprising a modulator 330 and a low pass filter 360. The modulators 320 and 330 are coupled to an oscillator 340. The arrangement of modulators 320, 330 coupled to oscillator 340 with low pass filters 350, 360 arranged behind the modulators 320, 330 is provided to implement a quadrature demodulation which allows to measure the real part I

$$I = Re ( BIOZ ) = a ( ω ) + k \cdot ( R {2}^{2} - R {1}^{2} ) \cdot {cin}^{2} \cdot {ω}^{2}$$

and the imaginary part Q

$$Q = Im ( BIOZ ) = b ( ω ) + k \cdot ( R 2 - R 1 ) \cdot cin \cdot ω$$

of the measured bioimpedance BIOZ.

For real part I of the measured bioimpedance BIOZ, based on the assumption of weekly frequency dependency of portion (a(w)) of real part I, it is proposed to estimate the derivative dI of real part I, and from that reconstruct the undesired real part contribution which is marked in bold letters in the equation of Re(BIOZ) given above. The derivative dI of the real part I can be estimated by subtracting the measured imaginary parts I1, I2 of two measurements at two consecutive frequencies ω1, ω2 among the available frequencies:

$$dI = \frac{I 2 - I 1}{ω 2 - ω 1} \approx 2 \cdot k \cdot ( R {2}^{2} - R {1}^{2} ) \cdot {cin}^{2} \cdot ω \approx 2 \cdot k \cdot ( R {2}^{2} - R {1}^{2} ) \cdot {cin}^{2} \cdot \frac{ω 2 + ω 1}{2}$$

The correction term will be then:

$$k \cdot ( R {2}^{2} - R {1}^{2} ) \cdot {cin}^{2} \cdot {ω}^{2} = \frac{d I}{ω 2 + ω 1} \cdot {ω}^{2}$$

Consequentyl, the corrected real Icorr of the measured bioimpedance is

$${I}_{corr} = I - \frac{d I}{ω 2 + ω 1} \cdot {ω}^{2}$$

where ω1 and ω2 are two frequencies near to the frequency F of interest; one of them could be the frequency of interest itself.

For imaginary part Q of the measured bioimpedance BIOZ, based on the assumption that the impedance is mostly real and the small imaginary part decreases with frequency, then another measurement of the bioimpedance can be performed at some high frequency ω3, higher than the frequency F of interest, and the imaginary part Q3 of the bioimpedance at frequency ω3 can be determined. This imaginary part Q3 will be a good approximation of the undesired part in the equation of Im(BIOZ) given above:

$$Q 3 = Q ( ω 3 ) = k \cdot ( R 2 - R 1 ) \cdot cin \cdot ω3$$

The correction term will then be:

$$k \cdot ( R 2 - R 1 ) \cdot cin = \frac{Q 3}{ω 3}$$

Consequently, the corrected imaginary part is:

$${Q}_{corr} = Q - \frac{Q 3}{ω 3} \cdot ω$$

Referring to FIG. 3, the bioimpedance measurement circuit comprises a signal processing circuit 400 for determining a corrected value of the bioimpedance (BIOZ), i.e. for determining the corrected real part Icorr and the corrected imaginary part Qcorr. The control circuit 200 is configured to control the measurement of the bioimpedance BIOZ by applying the stimulus current iin with a measurement frequency F to the electrode arrangement 100.

The signal processing circuit 400 is configured to determine the corrected value of the bioimpedance by calculating the corrected real part Icorr of the bioimpedance BIOZ in dependence on a derivative dI of the real part I of the bioimpedance BIOZ.

According to a possible embodiment, the signal processing circuit 400 is configured to determine the corrected real part Icorr of the bioimpedance by subtracting a first correction term, i.e. the term

$$\frac{d I}{ω2 + ω1} \cdot {ω}^{2} ,$$

from the real part I of the bioimpedance BIOZ determined by the evaluation circuit at the measurement frequency F. The first correction term is determined by the signal processing circuit 400 in dependence on the derivative dI of the real part I of the bioimpedance BIOZ.

The signal processing circuit 400 is further configured to determine a corrected imaginary part Qcorr of the bioimpedance BIOZ in dependence on the imaginary part Q of the bioimpedance BIOZ determined by the evaluation circuit at measurement frequency F of stimulus current iin, and an imaginary part Q3 of the bioimpedance determined by the evaluation circuit 300 determined at another measurement frequency ω3 of the stimulus current iin, wherein measurement frequency ω3 is higher than measurement frequency F of interest.

According to a possible embodiment, the signal processing circuit 400 is configured to determine the corrected imaginary part Qcorr of the bioimpedance by subtracting a second correction term, i.e. the term

$$\frac{Q 3}{ω 3} \cdot ω ,$$

from the imaginary part Q of the bioimpedance measured at the measurement frequency F of interest. The second correction term is determined in dependence on the imaginary part Q3 of the bioimpedance determined by the evaluation circuit 300 at the other measurement frequency ω3.

The control circuit 200 is configured to control the determination of the corrected value of the bioimpedance by carrying out at least four measurements of the bioimpedance BIOZ at four different measurement frequencies ω1, ω2, ω3, and F, wherein F is the frequency of interest. To apply the stimulus current iin to the electrode arrangement 100 with the at least four different measurement frequencies ω1, ω2, ω3, and F, the bioimpedance measurement circuit comprises a controllable stimulus current generator 500. Referring to FIG. 3, the controllable stimulus current generator 500 may be realized, as an example, by a controllable current source 501 connected to electrode 110, and a controllable current source 502 connected to electrode 120 of the electrode arrangement 100.

A first and second measurement of the bioimpedance is performed by the control circuit 200 to determine the derivative dI of the real part of the bioimpedance which is used to determine the first correction term for calculating the corrected real part Icorr of the bioimpedance.

The control circuit 200 is configured to control the controllable stimulus current generator 500 so that the stimulus current iin is applied to the electrode arrangement 100 with measurement frequency ω1 during the first measurement of the bioimpedance. The evaluation circuit 300 is configured to determine real part I1 of the bioimpedance BIOZ during the first measurement of the bioimpedance.

The control circuit 200 is further configured to control the controllable stimulus current generator 500 so that the stimulus current iin is applied to the electrode arrangement 100 with measurement frequency ω2 during the second measurement of the bioimpedance. The evaluation circuit 300 is configured to determine real part I2 of the bioimpedance BIOZ during the second measurement of the bioimpedance

Frequencies ω1 and ω2 are choosen to be near the frequency F of interest. According to a possible embodiment, one of frequencies ω1 and ω2 may be the frequency of interest itself.

The signal processing circuit 400 is configured to determine the derivative dI of the real part of the bioimpedance BIOZ by determining a quotient of the difference of real part I1 of the bioimpedance at measurement frequency ω1 and real part I2 of the bioimpedance at measurement frequency ω2, i.e. I1-I2, and the difference of the first and second measurement frequencies ω1-ω2, i.e.

$$dI = \frac{I 2 - I 1}{ω2 - ω1} .$$

The first correction term is then calculated by the signal processing circuit 400 as

$$\frac{d I}{ω 2 + ω 1} \cdot {ω}^{2} ,$$

wherein ω is equal to measurement frequency F of interest.

A third measurement of the bioimpedance is performed by the control circuit 200 to determine imaginary part Q3 of the bioimpedance which is used to determine the second correction term for calculating the corrected imaginary part Qcorr of the bioimpedance.

The control circuit 200 is configured to control the controllable stimulus current generator 500 so that the stimulus current iin is applied to the electrode arrangement 100 with measurement frequency ω3 during a third measurement of the bioimpedance. The evaluation circuit 300 is configured to determine imaginary part Q3 of the bioimpedance BIOZ during the third measurement of the bioimpedance. The second correction term is then calculated by the signal processing circuit 400 as

$$\frac{Q 3}{ω 3} \cdot ω ,$$

wherein ω is equal to measurement frequency F of interest.

If frequency ω3 is choosen to be higher than frequency ω or F of interest, the measured imaginary part Q3 will be a good approximation of the undesired imaginary part.

The corrected value of the bioimpedance BIOZ may then be calculated during a fourth measurement of the bioimpedance at frequency F of interest by calculating the corrected real part Icorr and the corrected imaginary part Qcorr of the bioimpedance.

The control circuit 200 is configured to control the controllable stimulus current generator 500 so that the stimulus current iin is applied to the electrode arrangement 100 with measurement frequency F during the fourth measurement of the bioimpedance. The evaluation circuit 300 is configured to determine the real part I and the imaginary part Q of the bioimpedance BIOZ during the fourth measurement of the bioimpedance.

The signal processing circuit 400 is configured to determine the corrected real part Icorr of the bioimpedance by subtracting the first correction term

$$\frac{d I}{ω 2 + ω 1} \cdot {ω}^{2}$$

from the real part I of the bioimpedance measured during the fourth measurement at measurement frequency ω=F, i.e.

$${I}_{corr} = I - \frac{d I}{ω2 + ω1} \cdot {ω}^{2} .$$

The signal processing circuit 400 is further configured to determine the corrected imaginary part Qcorr of the bioimpedance by subtracting the second correction term

$$\frac{Q 3}{ω 3} \cdot ω$$

from the imaginary part Q of the bioimpedance during the fourth measurement at measurement frequency ω=F, i.e.

$${Q}_{corr} = Q - \frac{Q 3}{ω 3} \cdot ω .$$

The embodiments of the bioimpedance measurement circuit disclosed herein have been discussed for the purpose of familiarizing the reader with novel aspects of the bioimpedance measurement circuit. Although preferred embodiments have been shown and described, many changes, modifications, equivalents and substitutions of the disclosed concepts may be made by one having skill in the art without unnecessarily departing from the scope of the claims.

In particular, the design of the bioimpedance measurement circuit is not limited to the disclosed embodiments, and gives examples of many alternatives as possible for the features included in the embodiments discussed. However, it is intended that any modifications, equivalents and substitutions of the disclosed concepts be included within the scope of the claims which are appended hereto.

Features recited in separate dependent claims may be advantageously combined. Moreover, reference signs used in the claims are not limited to be construed as limiting the scope of the claims.

Furthermore, as used herein, the term “comprising” does not exclude other elements. In addition, as used herein, the article “a” is intended to include one or more than one component or element, and is not limited to be construed as meaning only one.

This patent application claims the priority of German patent application with application No. 102022119225.3 and German patent application with application No. 102022122957.2, the disclosure contents of which are hereby incorporated by reference.

### REFERENCES

10 bioimpedance measurement circuit
100 electrode arrangement
110, 120, 130, 140 electrodes
200 control circuit
300 evaluation circuit
310 differential amplifier
320 modulator
330 modulator
340 oscillator
350 low pass filter
360 low pass filter
400 signal processing circuit
500 controllable stimulus current generator
501, 502 controllable current source
R1, R2, R3, R4 electrode contact impedance
BIOZ bioimpedance
I real part of bioimpedance
Q imaginary part of bioimpedance
Icorr corrected real part of bioimpedance
Qcorr corrected imaginary part of bioimpedance

1. A bioimpedance measurement circuit, comprising:
an electrode arrangement for attaching to a body,
an evaluation circuit for determining a real part and an imaginary part of a bioimpedance of the body in response to a stimulus current applied to the body and an input voltage of the evaluation circuit,
a signal processing circuit for determining a corrected value of the bioimpedance,
wherein the electrode arrangement includes a first pair of electrodes to apply the stimulus current to the body, and a second pair of electrodes to measure the input voltage in dependence on a voltage drop across the bioimpedance between the electrodes of the second pair of the electrodes, and
wherein the signal processing circuit is configured to determine the corrected value of the bioimpedance by calculating a corrected real part of the bioimpedance in dependence on a derivative of the real part of the bioimpedance, and a corrected imaginary part of the bioimpedance in dependence on the imaginary part and a second imaginary part of the bioimpedance determined by the evaluation circuit at different measurement frequencies.

2. The bioimpedance measurement circuit of claim 1,
wherein the signal processing circuit is configured to determine the corrected real part of the bioimpedance by subtracting a first correction term from the real part of the bioimpedance.

3. The bioimpedance measurement circuit of claim 2,
wherein the signal processing circuit is configured to determine the first correction term in dependence on the derivative of the real part of the bioimpedance.

4. The bioimpedance measurement circuit of claim 1,
wherein the signal processing circuit is configured to determine the corrected imaginary part of the bioimpedance by subtracting a second correction term from the imaginary part of the bioimpedance.

5. The bioimpedance measurement circuit of claim 4,
wherein the signal processing circuit is configured to determine the second correction term in dependence on the second imaginary part of the bioimpedance.

6. The bioimpedance measurement circuit of claim 1, comprising:
a control circuit to control a measurement of the bioimpedance, and
wherein the control circuit is configured to control a determination of the corrected value of the bioimpedance by carrying out at least four measurements of the bioimpedance at four different measurement frequencies.

7. The bioimpedance measurement circuit of claim 6, comprising:
a controllable stimulus current generator to apply the stimulus current to the electrode arrangement with the at least four different measurement frequencies.

8. The bioimpedance measurement circuit of claim 7,
wherein the control circuit is configured to control the controllable stimulus current generator so that the stimulus current is applied to the electrode arrangement with a first one of the measurement frequencies during a first one of the at least four measurements of the bioimpedance, and
wherein the evaluation circuit is configured to determine a second real part of the bioimpedance during the first measurement of the bioimpedance.

9. The bioimpedance measurement circuit of claim 7,
wherein the control circuit is configured to control the controllable stimulus current generator so that the stimulus current is applied to the electrode arrangement with a second one of the measurement frequencies during a second one of the at least four measurements of the bioimpedance, and
wherein the evaluation circuit is configured to determine a third real part of the bioimpedance during the second measurement of the bioimpedance.

10. The bioimpedance measurement circuit of claim 7,
wherein the control circuit is configured to control the controllable stimulus current generator so that the stimulus current is applied to the electrode arrangement with a third one of the measurement frequencies during a third one of the at least four measurements of the bioimpedance, and
wherein the evaluation circuit is configured to determine the second imaginary part of the bioimpedance during the third measurement of the bioimpedance.

11. The bioimpedance measurement circuit of claim 7,
wherein the control circuit is configured to control the controllable stimulus current generator so that the stimulus current is applied to the electrode arrangement with a fourth one of the measurement frequencies during a fourth one of the at least four measurements of the bioimpedance, and
wherein the evaluation circuit is configured to determine the real part and the imaginary part of the bioimpedance during the fourth measurement of the bioimpedance.

12. The bioimpedance measurement circuit of claim 11,
wherein the signal processing circuit is configured to determine the corrected real part of the bioimpedance by subtracting a first correction term from the real part of the bioimpedance during the fourth measurement, and
wherein the signal processing circuit is configured to determine the corrected imaginary part of the bioimpedance by subtracting a second correction term from the imaginary part of the bioimpedance during the fourth measurement.

13. The bioimpedance measurement circuit of claim 10,
wherein the signal processing circuit is configured to determine the derivative of the real part of the bioimpedance by determining a quotient of a difference of a second and third real part of the bioimpedance, and a difference of a first and second measurement frequencies.

14. The bioimpedance measurement circuit of claim 11,
wherein the control circuit is configured to control the controllable stimulus current generator so that a first and second frequencies are closer to a fourth frequency than a third frequency, and the third frequency is higher than the fourth frequency.

15. The bioimpedance measurement circuit of claim 1,
wherein the evaluation circuit is configured to determine the real part and the imaginary part of the bioimpedance by quadrature demodulation.
